(19) United States
(12) Patent Application Publication
(10) Pub. No.: US 2018/0296740 A1
(43) Pub. Date: Oct. 18, 2018

(54) NASAL IRRIGATOR

(71) Applicants: Yulia MARKMAN, Riverside, CA (US); Mohamed ABU HUSSIN, Riverside, CA (US)

(72) Inventors: Yulia MARKMAN, Riverside, CA (US); Mohamed ABU HUSSIN, Riverside, CA (US)

(21) Appl. No.: 15/488,152

(22) Filed: Apr. 14, 2017

(51) Int. Cl. A61M 1/00; A61M 11/00

(52) U.S. Cl. CPC A61M 1/0058; A61M 11/006; A61M 2210/0618; A61M 2205/0222; A61M 2205/587; A61M 2205/502; A61M 2205/0238; A61M 2205/59

(57) ABSTRACT

A nasal irrigation device and system is described that provides gentle and effective irrigation of the nasal cavity. Small volumes of irrigation fluid are directed towards the wall of the sinus cavity and collected centrally, using a pliable irrigation device. A focusing feature is provided that serves to hold the attention of children undergoing nasal irrigation in order to distract them from the procedure. Such a focusing feature can be in the form of a stuffed or toy animal that envelops or forms part of the nasal irrigation system.

The field of the invention is irrigation and aspiration devices, particularly nasal aspirators for use with children.

### BACKGROUND

The following description includes information that may be useful in understanding the present invention. It is not an admission that any of the information provided herein is prior art or relevant to the presently claimed invention, or that any publication specifically or implicitly referenced is prior art.

Nasal congestion is a well known and essentially universal health issue, and can result from allergies, viral or bacterial infections, and/or mechanical irritation. It is particularly common in infants and small children. Inflammation within the nasal cavity results in swelling and excessive production of mucus, which in turn makes breathing through the nose difficult. This is a source of discomfort, and prolonged nasal congestion can impede normal nasal function and lead to the development of sinus and ear infections. Nasal congestion is particularly problematic in small children, who rely on breathing through their nose while nursing and cannot voluntarily blow their nose. While nasal congestion can be treated pharmaceutically (for example, using decongestants and/or antihistamines) it is desirable in many circumstances (for example, small children or persons with hypertension) to avoid pharmaceutical intervention. Mechanical devices, such as nasal splints, have also been used to help open nasal airways. These devices, however, require a high degree of compliance and need to be correctly sized to function properly. As such they are not useful for small children.

One method of relieving the symptoms of nasal congestion in young children is to physically remove accumulated mucus by gentle suction. This is generally attempted using a flexible nozzle attached to a suction bulb, which is squeezed and release to generate the necessary suction. In some instances, such as U.S. Pat. No. 6,290,667 (to Cook), the suction bulb is disguised as an animal figure. All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. This approach is rarely effective, however, as nasal secretions are often too viscous to move freely. Adult caretakers often find that children associate the discomfort associated with removing these thickened secretions with the use of the suction device, and quickly learn to actively avoid its use. Attempts have been made to simplify operation of such devices. For example, International Patent Application Publication No. WO9425082A1 (to Brunet) describes a device that uses flexible probes attached to pumps to remove nasal mucus, and that provides an animal figure that serves to distract or amuse a child during use. It is not apparent, however, how such a device addresses removal of thickened or hardened mucus.

Another approach to relieving the symptoms of nasal congestion has been to introduce a liquid to the nasal cavity, and then withdraw the liquid along with loosened mucus. The “neti pot” is a well known traditional device for performing this operation. Use of a neti pot, however, requires learning the technique, coordination, and cooperation on the part of the individual being treated. More recently devices that simplify the process by providing a controlled, pressurized volume of liquid and a collection chamber (for example, the Navage™ Nasal Hygiene System, by RhinoSystems, Ohio). Such devices, however, are not recommended for use with children. This may be due, at least in part, to the large volumes of liquid used.

Devices have been developed for the introduction and removal of fluids to and from the human body. An example of such a device can be found in U.S. Pat. No. 6,342,061 (to Kauker), which describes an elongated, rigid device with openings along its length for dispensing and taking in an irrigating solution. It is not clear, however, if such an arrangement would permit adequate removal of hardened or thickened nasal mucus. In addition, while rigidity is advantageous for the specified medical use it is not suitable for nasal irrigation (particularly in small children).

Thus, there is still a need for a convenient, safe, and effective means of relieving nasal congestion and/or removing accumulated mucus from the nasal cavity.

### SUMMARY OF THE INVENTION

Embodiments of the inventive concept include methods, devices, and systems that provide an elongated, flexible irrigation device having one or more outlets that release an irrigation fluid outwards from the lateral wall of the irrigation device and that collected waste fluid from the terminus of the device. Outlet and inlet lumens are provided, and can occupy separate and distinct volumes within the irrigation device, or overlapping or partially overlapping volumes within the irrigation device. The irrigation device can be part of a nasal irrigation system that provides an irrigation fluid, a waste reservoir, and mechanisms for directed nasal irrigation fluid and waste fluid. The nasal irrigation system can include an attention focusing feature, such as a stuffed or toy animal figure or head, that is useful in distracting children when the nasal irrigation system is in use.

One embodiment of the inventive concept is a nasal irrigation device that include a pliant tube comprising a first lumen, a second lumen, a first aperture, a second aperture, and a terminus. The first aperture is positioned at the terminus and provides fluid communication between an external environment and the first lumen. The pliant tube is made of any suitable flexible or pliant material, for example rubber, latex, silicone rubber, and/or nitrile rubber, and can have an external surface that provides lubrication. The second aperture is oriented approximately normal to the first aperture and provides fluid communication between the second lumen and the external environment. In some embodiments the pliant tube has an outer tube that acts as the second lumen that at least partially encloses an inner tube that acts as the first lumen. In other embodiments the pliant tube has an outer tube that acts as the second lumen that at least partially encloses an inner tube that acts as the first lumen. In some embodiments the first lumen and the second lumen can occupy non-overlapping volumes within the nasal irrigation device, or can occupy volumes that partially or essentially completely overlap. In some embodiments the second lumen is in fluid communication with a source of irrigation solution, which can be pressurized. In some embodiments the first lumen is in fluid communication with a source of negative pressure. In such embodiments a collection reservoir can be placed between the first lumen and the source of negative pressure. In a preferred embodiment the pliant tube is dimensioned to lie within a nare or a nasal cavity of a human child.

10. The nasal irrigation device of claim 1, wherein the pliant tube comprises a pliant material selected from the group consisting of r.

Another embodiment of the inventive concept is a method of treating nasal congestion that includes providing an irrigation device as described above, applying, via the second aperture, a volume of an irrigation solution to the nasal cavity and withdrawing, via the first aperture, a waste fluid from the nasal cavity. In such a method the waste fluid can be directed to a collection reservoir. The volume of the irrigation solution can range from about 0.1 mL to about 5 mL or more, and can be applied as a droplet spray or as a stream of liquid. In some embodiments the steps of applying the irrigation solution and withdrawing the waste solution are performed sequentially. In other embodiments the waste solution can be withdrawn while the irrigation solution is being dispensed.

Another embodiment of the inventive concept is a nasal irrigation system that includes an irrigation device as described above, an irrigation fluid reservoir that is in fluid communication with the second lumen; and a waste fluid reservoir that is in fluid communication with the first lumen. In some embodiments the irrigation device is replaceable. The irrigation fluid reservoir can be a pliant envelope that holds the irrigation fluid. The nasal irrigation system can include a pressurizing mechanism directed to the irrigation fluid reservoir. Such a pressurizing mechanism can be a pump, or can be a device for applying mechanical pressure to the irrigation fluid reservoir. The nasal irrigation system can include a pressurizing mechanism that is in fluid communication with the waste fluid reservoir. Such a pressurizing mechanism can be a pump, or can be a vacuum reservoir.

In preferred embodiments the nasal irrigation system includes an attention focusing feature. Such an attention focusing feature can include a visual target that resembles a face. In some embodiments the attention focusing feature has a visual target that includes one or more lights, and/or an electronic display. In still other embodiments the attention focusing feature emits a sound.

The nasal irrigation system includes an irrigation fluid reservoir, which can further include an irrigation fluid. Such an irrigation fluid can be a saline solution, however in some embodiments the irrigation fluid can include a pharmaceutical compound.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 schematically depicts sectional views of an irrigation device of the inventive concept, where inlet and outlet lumens occupy separate and distinct volumes.

FIG. 2 schematically depicts sectional views of an irrigation device of the inventive concept, where inlet and outlet lumens occupy overlapping volumes.

FIG. 3 schematically depicts sectional views of an irrigation device of the inventive concept, where inlet and outlet lumens occupy partially overlapping volumes.

FIG. 4 schematically depicts sectional views of an irrigation device of the inventive concept, where outlet lumens connect to a common channel.

FIGS. 5A to 5E schematically depict nasal irrigation systems of the inventive concept. FIG. 5A shows a generic nasal irrigation system, having an irrigation fluid reservoir and waste reservoir, with associated feedlines in fluid communication with an irrigation device. FIG. 5B shows a nasal irrigation system having a pump positioned between the irrigation fluid reservoir and the irrigation device. FIG. 5C depicts a nasal irrigation system having a pump positioned to pressurize an irrigation fluid reservoir to impel irrigation fluid towards an irrigation device. FIG. 5D depicts a nasal irrigation system having a pump positioned between the irrigation device and the waste reservoir. FIG. 5E depicts an nasal irrigation system having a pump positioned to apply negative pressure to the waste reservoir, which in turn applied negative pressure to the irrigation device.

### DETAILED DESCRIPTION

The inventive subject matter provides apparatus, systems and methods in which an elongated flexible nozzle having one or more outlet lumen(s) that deliver(s) an irrigation solution from the side (e.g. laterally) of the nozzle and one or more inlet lumen(s) that take up the dispensed irrigation solution is provided. The nozzle is introduced into the nasal cavity of a subject in need of nasal irrigation, and a volume (typically 5 mL or less) of an irrigation solution is introduced using the outlet lumen(s). The positioning of the opening from the outlet lumen directs the irrigation solution towards to walls of the sinus cavity, where it is highly effective in dislodging and/or reducing the viscosity of mucus within the sinus cavity. The dispensed irrigation solution is subsequently taken up by the inlet lumen, which has an opening at or near the tip of the nozzle. This positioning of outlet and inlet lumens permits gentle but thorough removal of accumulated mucus and subsequent relief of the symptoms of nasal congestion.

In a preferred embodiment the elongated flexible nozzle is part of a nasal irrigation system, which includes a reservoir of irrigation solution and a waste reservoir that receives the spent irrigation fluid and dislodged mucus. The elongated flexible nozzle can be removable, permitting the use of different nozzles with different individuals and/or disposal of contaminated nozzles. Such a system can include a mechanism or device to pressurize the irrigation solution in order to provide fluid flow through the outlet lumen(s). Such a system can also include a mechanism or device to provide reduced pressure to the inlet lumen in order to provide a fluid flow through the inlet lumen and to the waste reservoir. In a preferred embodiment such a system includes a focusing feature, such as a toy figure, electronic display, light display, and/or sound device. Such a focusing feature serves to distract a subject on whom the system is being used, and can aid in proper positioning and/or orientation of the head during use.

One should appreciate that devices, systems, and methods of the inventive concept provide a safe and effective means of relieving nasal congestion without pharmaceutical intervention, and do so in a manner that is highly compatible with small children.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

One embodiment of the inventive concept is an irrigation device for use in a nasal cavity (for example, a nasal cavity of a child). Such an irrigation device can have an elongate shape that tapers, and can be made of a pliable or flexible material. Suitable materials include rubber, latex, silicone rubber, nitrile, or any biocompatible and suitably pliant and water resistant material. The narrow end of the irrigation device can include an opening that leads to an inlet passage or lumen, through which waste irrigation liquid and/or loosened mucus (or other body fluids) pass when the irrigation device is in use. The irrigation device also includes one or more outlet lumens, which have outlet openings along the tapered outer wall of the irrigation device. Such an outlet lumen provides a flow or spray of irrigation fluid when in use, and the position of the outlet opening directs the flow or spray is directed essentially normal to the outer surface of the irrigation device. In some embodiments, the inlet lumen and the outlet lumen are separate and spatially distinct (i.e. have non-overlapping volumes). In other embodiments the inlet lumen and the outlet lumen have volumes that overlap. For example, all or a portion of an inlet lumen can lie completely within all or within of the volume of an outlet lumen (e.g. in a tube within a tube arrangement), or vice versa. In other embodiments the inlet lumen and the outlet lumen have volumes that partially overlap. For example, an outlet lumen can be imbedded in a wall of an inlet lumen, or vice versa.

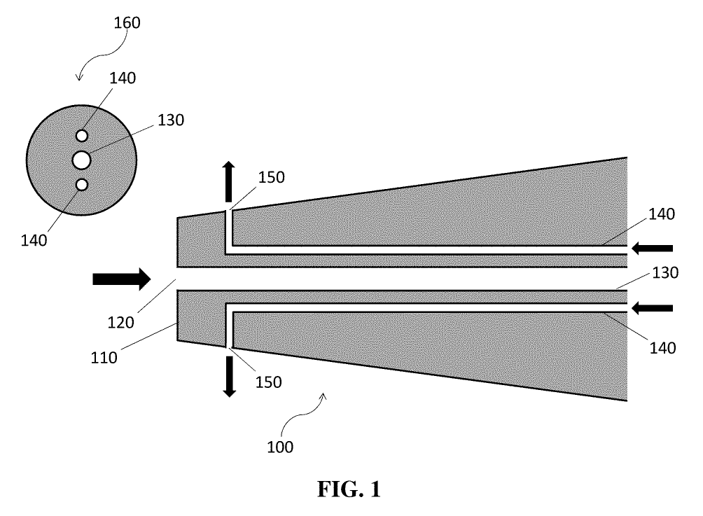

An example such an irrigation device in which the inlet and outlet lumens do not occupy overlapping volumes is shown in FIG. 1, where arrows indicate the direction of fluid flow when in use. The irrigation device 100 has an elongated tapered shape that ends in a terminus 110. The terminus 110 includes an inlet opening or aperture 120 that provides a fluid inlet for an inlet lumen 130, which serves to remove spent irrigation fluid and loosened mucus or other fluids. The irrigation device can include one or more outlet lumen(s) 140, which occupy a different and distinct volume(s) than the inlet lumen 130 within the irrigation device 100. This can be seen in cross section, which is shown as an inset 160. An outlet lumen, which serves to introduce an irrigation fluid, can have one or more outlet opening(s) or aperture(s) 150 that is directed at approximately a right angle (±30° from normal) to the major axis of the inlet lumen 130 or to the angle of the outer surface of the irrigation device 100. It should be appreciated that, while an outlet lumen 140 is shown as incorporating an angle or bend, such an outlet lumen can be essentially straight and positioned within the irrigation device 100 to intersect the tapered wall of the irrigation device in order to provide an outlet aperture 150. This arrangement directs the outgoing flow of irrigation fluid towards the walls of the nasal cavity when in use. Positioning of the inlet aperture 120 at the terminus of the irrigation device 100 provides mixing of the irrigation fluid with mucus secretions and other body fluids prior to aspiration. The body of the irrigation device 100 is preferably constructed of a flexible or pliant material (e.g. rubber, latex, silicone rubber, nitrile, etc.) that permits it to bend and otherwise conform to the nasal cavity.

An example such an irrigation device in which the inlet and outlet lumens do not occupy overlapping volumes is shown in FIG. 1, where arrows indicate the direction of fluid flow when in use. The irrigation device 100 has an elongated tapered shape that ends in a terminus 110. The terminus 110 includes an inlet opening or aperture 120 that provides a fluid inlet for an inlet lumen 130, which serves to remove spent irrigation fluid and loosened mucus or other fluids. The irrigation device can include one or more outlet lumen(s) 140, which occupy a different and distinct volume(s) than the inlet lumen 130 within the irrigation device 100. This can be seen in cross section, which is shown as an inset 160. An outlet lumen, which serves to introduce an irrigation fluid, can have one or more outlet opening or aperture 150 that is directed at approximately a right angle (±30° from normal) to the major axis of the inlet lumen 130 or to the angle of the outer surface of the irrigation device 100. This arrangement directs the outgoing flow of irrigation fluid towards the walls of the nasal cavity when in use. Positioning of the inlet aperture 120 at the terminus of the irrigation device 100 provides mixing of the irrigation fluid with mucus secretions and other body fluids prior to aspiration. The body of the irrigation device 100 is preferably constructed of a flexible or pliant material (e.g. rubber, latex, silicone rubber, nitrile, etc.) that permits it to bend and otherwise conform to the nasal cavity.

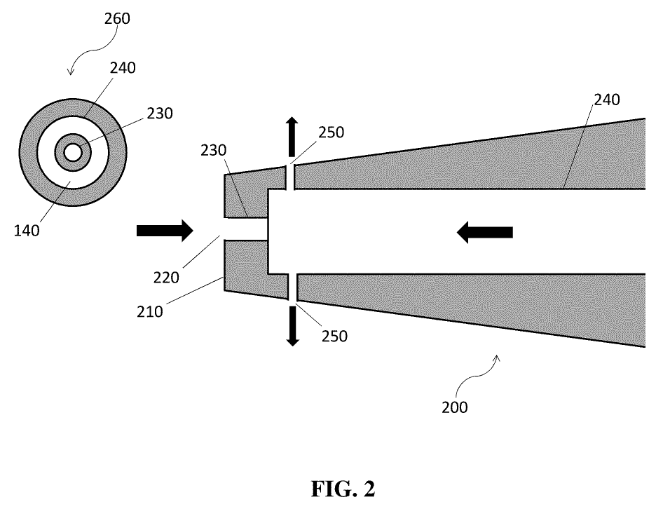

An example such an irrigation device in which the inlet and outlet lumens occupy overlapping volumes is shown in FIG. 2, where arrows indicate the direction of fluid flow when in use. The irrigation device 200 has an elongated tapered shape that ends in a terminus 210. The terminus 210 includes an inlet opening or aperture 220 that provides a fluid inlet for an inlet lumen 230, which serves to remove spent irrigation fluid and loosened mucus or other fluids. The irrigation device can include one or more outlet lumen(s) 240, partially or completely envelop the inlet lumen 230 within the irrigation device 200. This can be seen in cross section, which is shown as an inset 260. Such an outlet lumen, which serves to introduce an irrigation fluid, can have one or more outlet opening or aperture 250 that is directed at approximately a right angle (±30° from normal) to the major axis of the inlet lumen 230 or to the angle of the outer surface of the irrigation device 200. This arrangement directs the outgoing flow of irrigation fluid towards the walls of the nasal cavity when in use. Positioning of the inlet aperture 220 at the terminus of the irrigation device 200 provides mixing of the irrigation fluid with mucus secretions and other body fluids prior to aspiration. It should be appreciated that, while an outlet lumen 240 is shown as incorporating an angle or bend, such an outlet lumen can be essentially straight and positioned within the irrigation device 200 to intersect the tapered wall of the irrigation device in order to provide an outlet aperture 250. The body of the irrigation device 200 is preferably constructed of a flexible or pliant material (e.g. rubber, latex, silicone rubber, nitrile, etc.) that permits it to bend and otherwise conform to the nasal cavity.

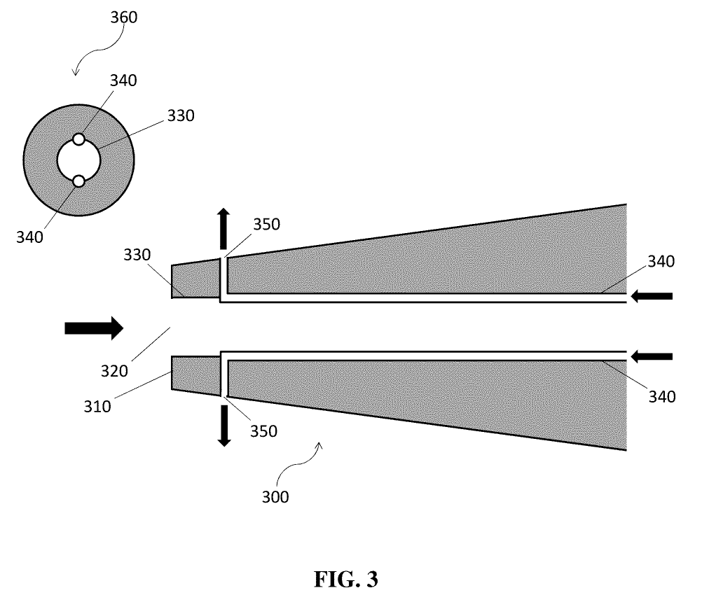

An example such an irrigation device in which the inlet and outlet lumens occupy partially overlapping volumes is shown in FIG. 3, where arrows indicate the direction of fluid flow when in use. The irrigation device 300 has an elongated tapered shape that ends in a terminus 310. The terminus 310 includes an inlet opening or aperture 320 that provides a fluid inlet for an inlet lumen 330, which serves to remove spent irrigation fluid and loosened mucus or other fluids. The irrigation device can include one or more outlet lumen(s) 340, partially or completely imbedded in a wall of the inlet lumen 330 within the irrigation device 300. This can be seen in cross section, which is shown as an inset 360. Such an outlet lumen, which serves to introduce an irrigation fluid, can have one or more outlet opening or aperture 350 that is directed at approximately a right angle (±30° from normal) to the major axis of the inlet lumen 330 or to the angle of the outer surface of the irrigation device 300. This arrangement directs the outgoing flow of irrigation fluid towards the walls of the nasal cavity when in use. It should be appreciated that, while an outlet lumen 340 is shown as incorporating an angle or bend, such an outlet lumen can be essentially straight and positioned within the irrigation device 300 to intersect the tapered wall of the irrigation device in order to provide an outlet aperture 350. Positioning of the inlet aperture 320 at the terminus of the irrigation device 300 provides mixing of the irrigation fluid with mucus secretions and other body fluids prior to aspiration. The body of the irrigation device 300 is preferably constructed of a flexible or pliant material (e.g. rubber, latex, silicone rubber, nitrile, etc.) that permits it to bend and otherwise conform to the nasal cavity.

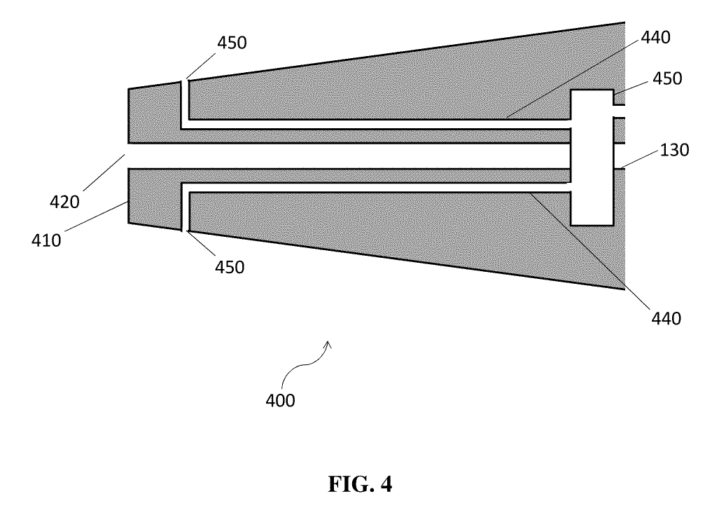

In some embodiments the irrigation device can include outlet lumens that terminate in a common channel, which is serves to provide a common source for irrigation fluid that is to be dispensed. An example of such an embodiment is shown in FIG. 4. The irrigation device 400 has an elongated tapered shape that ends in a terminus 410. The terminus 410 includes an inlet opening or aperture 420 that provides a fluid inlet for an inlet lumen 430, which serves to remove spent irrigation fluid and loosened mucus or other fluids. The irrigation device can include one or more outlet lumen(s) 440, shown here as occupying separate and distinct volumes from the inlet lumen 430. An outlet lumen 440 can be in fluid communication with a channel 450. Such a channel 450 can extend around all or a portion of the irrigation device, and can serve as an inlet for irrigation fluid being supplied to the irrigation device, simplifying coupling of the irrigation device to a source of irrigation fluid. As shown, two (or more) outlet lumens 440 can be in fluid communication with such a channel 450, thereby allowing a single feed of irrigation fluid to provide irrigation fluid for multiple outlet lumens. Such an outlet lumen, which serves to introduce an irrigation fluid, can have one or more outlet opening or aperture 450 that is directed at approximately a right angle (±30° from normal) to the major axis of the inlet lumen 430 or to the angle of the outer surface of the irrigation device 400. This arrangement directs the outgoing flow of irrigation fluid towards the walls of the nasal cavity when in use. Positioning of the inlet aperture 420 at the terminus of the irrigation device 400 provides mixing of the irrigation fluid with mucus secretions and other body fluids prior to aspiration. The body of the irrigation device 400 is preferably constructed of a flexible or pliant material (e.g. rubber, latex, silicone rubber, nitrile, etc.) that permits it to bend and otherwise conform to the nasal cavity.

Another embodiment of the inventive concept is a nasal irrigation system that incorporates an irrigation device as described above. In such a system the irrigation device can be fixed or removable. In a preferred embodiment the irrigation device is removable, to allow for replacement due to wear, use of different irrigation devices by different individuals using the same system, and/or for sanitary purposes. Such a nasal irrigation system can be configured to fit in the hand of a user and/or a person applying nasal irrigation, or can be desktop, benchtop, or mounted unit that is in fluid communication with an associated irrigation device (for example, through the use of flexible tubes).

Such a nasal irrigation system can include a reservoir of irrigation fluid, a device for motivating the irrigation fluid, a waste storage area for collection of a waste stream that includes waste irrigation fluid and/or loosened mucus or other body fluids, a mechanism for motivating the waste stream towards the waste storage area, and, optionally, a focusing feature.

Such a system can be supplied with an irrigation fluid. An irrigation fluid can be a biocompatible liquid formulation, for example an aqueous solution of approximately physiological ionic strength and pH. In a preferred embodiment the irrigation fluid is a saline solution, which can be buffered to a pH of approximately 6 to 8. In other embodiments the irrigation solution can include a pharmaceutical compound, for example an antihistamine, decongestant, antibacterial, and/or antiviral compound. Such an irrigation fluid can be provided in bulk, and added to a reservoir of the nasal irrigation system as needed. Alternatively, irrigation fluid can be provided as single or multiple use ampoules or cartridges.

Irrigation fluid can be stored in any suitable container or enclosure, either onboard the nasal irrigation system or in fluid communication with it. In some embodiments the irrigation fluid is provided in a rigid container. In such embodiments the irrigation fluid can be moved through an outlet lumen through the application of pressure, for example air pressure within the irrigation fluid reservoir or through the use of a pump (e.g. a diaphragm, peristaltic, piston, or rotary pump). In other embodiments the irrigation fluid can be provided in a pliant container. In such an embodiment the irrigation fluid can be moved through an outlet lumen through the application of mechanical force to the pliant container sufficient to deform the container (for example, through squeezing via a handgrip).

As noted above, such a nasal irrigation system can provide a waste reservoir for collection of a waste stream, such as waste irrigation fluid and/or loosened mucus or other body fluids. In some embodiments such a waste reservoir can be transparent, permitting a user with a visual indication of the need to empty and/or clean the waste reservoir. Fluid flow into the inlet lumen and to the waste reservoir can be passive (e.g. without the use of a dedicated mechanism of the nasal irrigation system), for example by gravity and/or exhalation of a subject undergoing nasal irrigation. In other embodiments the nasal irrigation system can apply negative pressure to the inlet lumen in order to collect a waste stream. Such negative pressure can be provided, for example, by a vacuum reservoir (such as a squeeze bulb or bottle) that is in fluid communication with the waste reservoir. In other embodiments the nasal irrigation system can include a pump (e.g. a diaphragm, peristaltic, piston, or rotary pump) that applies negative pressure to the inlet lumen. Such a pump can be positioned between the inlet lumen and the waste reservoir, or can be positioned past the waste reservoir (in order to minimize contamination).

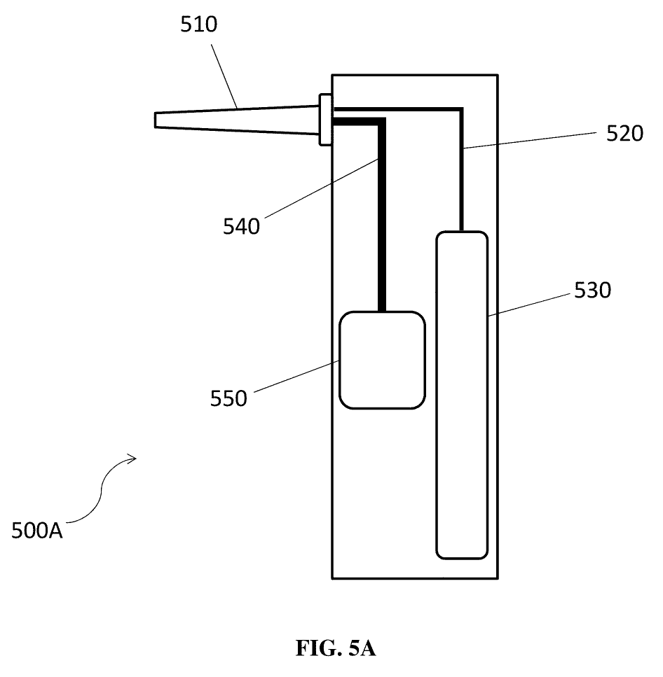

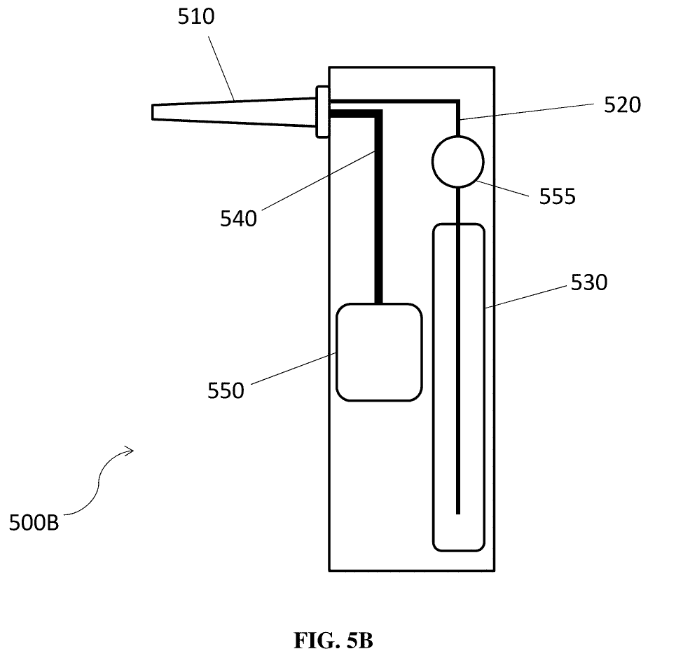

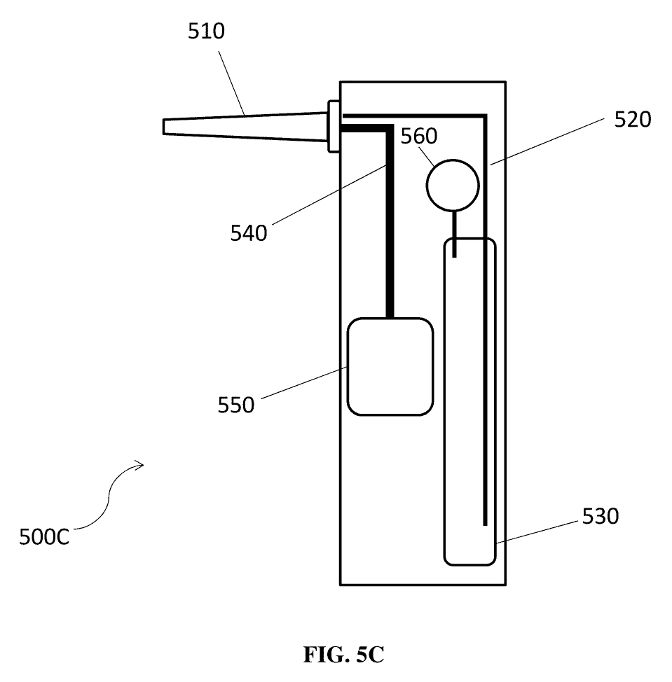

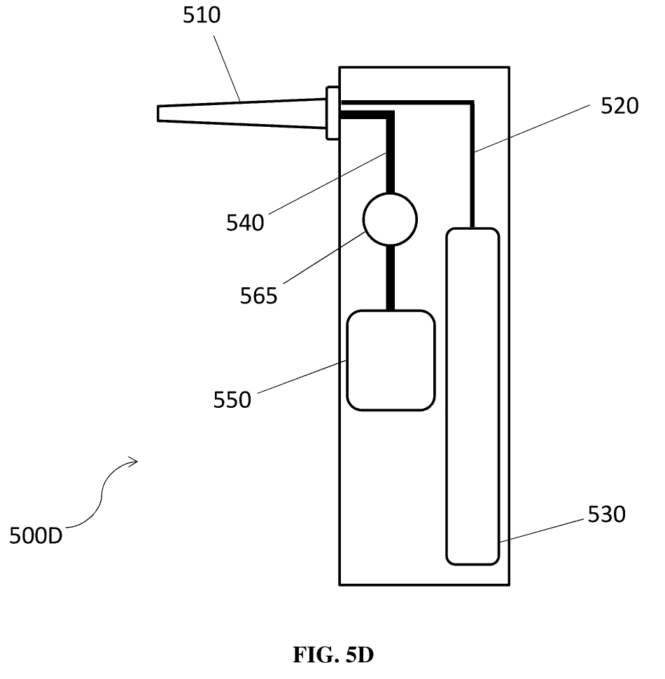

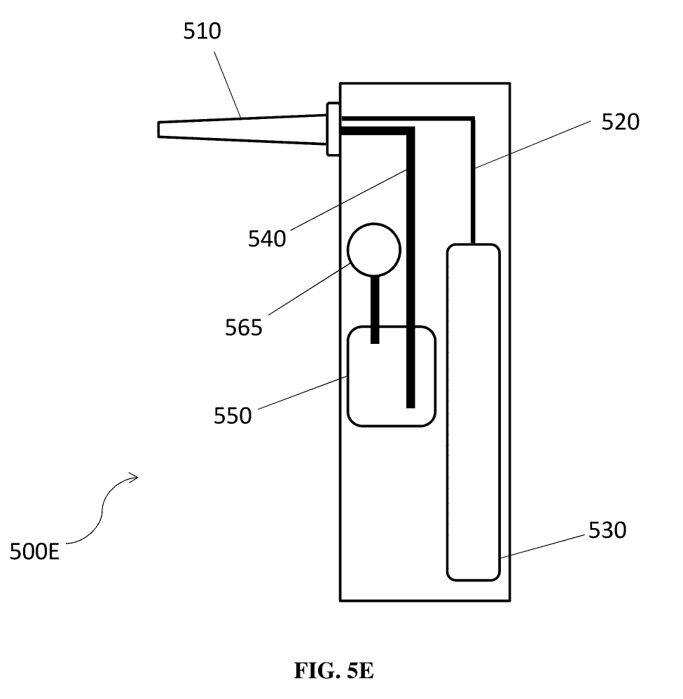

Examples of nasal irrigation systems of the inventive concept can be seen in FIGS. 5A to 5E. FIG. 5A schematically depicts a generic nasal irrigation system 500A having an irrigation device 510, which is in turn in fluid communication with an irrigation fluid reservoir 530 via an irrigation fluid line 520, and is in fluid communication with a waste reservoir 550 via a waste fluid line 550. It should be appreciated that a nasal irrigation system of the inventive concept can incorporate some or all of the features described in FIGS. 5B to 5E, in any combination.

FIG. 5B schematically depicts a nasal irrigation system 500B having an irrigation device 510, which is in turn in fluid communication with an irrigation fluid reservoir 530 via an irrigation fluid line 520. A pump 555 (e.g. a diaphragm, peristaltic, piston, or rotary pump) is placed inline with the irrigation fluid line 520, and withdraws irrigation fluid from the irrigation fluid reservoir 530 and dispenses it to the irrigation device 510. The irrigation device 510 is also in fluid communication with a waste reservoir 550 via a waste fluid line 550.

An alternative arrangement is shown in FIG. 5C, which schematically depicts a nasal irrigation system 500C having an irrigation device 510, which is in turn in fluid communication with an irrigation fluid reservoir 530 via an irrigation fluid line 520. A pump 560 (e.g. a diaphragm, peristaltic, piston, or rotary pump) is placed in fluid communication with the irrigation fluid reservoir 520, and applies pressure to the irrigation fluid reservoir 530 to drive irrigation fluid through the irrigation fluid line 520 for dispensing through the irrigation device 510. The irrigation device 510 is also in fluid communication with a waste reservoir 550 via a waste fluid line 550.

Another embodiment is shown in FIG. 5D, which schematically depicts a nasal irrigation system 500D having an irrigation device 510, which is in turn in fluid communication with an irrigation fluid reservoir 530 via an irrigation fluid line 520. The irrigation device 510 is also in fluid communication with a waste reservoir 550 via a waste fluid line 540. A pump 565 (e.g. a diaphragm, peristaltic, piston, or rotary pump) is placed in fluid communication with the waste fluid line 540 between the waste reservoir 550 and the irrigation device 510, and applies negative pressure to an inlet lumen of the irrigation device 510 through the waste fluid line 540 in order to direct waste fluid and/or loosened mucus or other body fluids for collection in the waste reservoir 550.

In an alternative arrangement, shown in FIG. 5E, which schematically depicts a nasal irrigation system 500E having an irrigation device 510, which is in turn in fluid communication with an irrigation fluid reservoir 530 via an irrigation fluid line 520. The irrigation device 510 is also in fluid communication with a waste reservoir 550 via a waste fluid line 540. A pump 570 (e.g. a diaphragm, peristaltic, piston, or rotary pump) is placed in fluid communication with the irrigation fluid reservoir 520, which in turn applies negative pressure to an inlet lumen of the irrigation device 510 through the waste fluid line 540 for collection in the waste reservoir 550. Such an arrangement advantageously reduces fouling of the pump 570.

As noted above, the devices and systems described herein are preferably intended for use with children. Such children often cannot appreciate that nasal irrigation is helpful and can reduce discomfort, and may resist use of the nasal irrigation system. Towards that end, in preferred embodiments of the inventive concept the nasal irrigation system includes an attention focusing feature. Such a feature can essentially envelop the device, disguising the system. In other embodiments a focusing feature can occupy a portion of the device, for example being placed at or near the nozzle of the irrigation device. The distracting feature serves to capture the attention of a child undergoing treatment, distracting them from the procedure. It can also serve to properly maintain the position of the child's head as they maintain eye contact with the distracting feature.

Such an attention focusing feature can, for example, be configured as a toy or stuffed animal or animal head. In a preferred embodiment such a toy or stuffed animal can incorporate a feature that is complementary with the irrigation nozzle. For example, the trunk of a toy or stuffed animal head can be used to disguise the nozzle portion of the device. A distracting feature can include one or more lights, which can, in some embodiments, flash or otherwise provide a distracting light pattern. Similarly, a distracting feature can include an electronic display. This can provide a distracting image or video feed, and in some embodiments can include a timer display useful to the individual administering treatment. In still other embodiments the distracting feature can emit one or more sounds, which can be selected at the discretion of the individual administering treatment. Such sounds can serve as a stimulus to focus attention away from the irrigation process, and/or can be used as a positive reinforcement. It should be appreciated that provision of such sounds can be particularly useful when the nasal irrigation system is utilized with visually impaired children.

Another embodiment of the inventive concept is a method for irrigation a nasal cavity using a device or system as described above. In such a method a flexible tapered irrigation device of a nasal irrigation system is introduced into a nare or nasal cavity of an individual in need of nasal irrigation. An attention focusing feature of the nasal irrigation system can be engaged to draw the attention of the individual being treated in order to distract them from the irrigation process and/or aid in proper positioning of their head. A small volume (e.g. about 0.1 mL. 0.2 mL, 0.3 mL, 0.4 mL, 0.5 mL, 0.8 mL, 1 mL, 1.3 mL, 1.5 mL, 1.8 mL, 2 mL, 2.5 mL, 3 mL, 3.5 mL, 4 mL, 4.5 mL, 5 mL, or greater than 5 mL) of an irrigation fluid is introduced into the nasal cavity through apertures that are positioned along the outer wall of the irrigation device, and that direct the irrigation fluid towards the interior walls of the nare and/or sinus cavity. Such volumes can be delivered at once, or as a series of smaller applications. The irrigation fluid can be provided as a suspension of liquid droplets (e.g. a mist or aerosol), as a stream, or can transition between a suspension of liquid droplets and a stream during application. Irrigation fluid can be applied by activating a pump that is in fluid communication with a reservoir of irrigation fluid. In some embodiments irrigation is applied by applying mechanical pressure (e.g. via a push button, hand grip, trigger, or similar device) to a flexible reservoir containing the irrigation fluid. Such mechanical pressure can be applied manually or via a motor or other powered device. In such embodiments the irrigation fluid can be supplied as single or multiple use ampoules constructed of a pliant or compressible materials. In some embodiments irrigation fluid is provided in an ampoule that incorporates a movable piston that includes a movable seal (e.g. an o-ring) that engages one or more rigid wall of the ampoule. In such an embodiment advancement of the piston can provide delivery of controlled amounts of irrigation fluid at a controlled rate.

Application of the irrigation fluid thins and/or dislodges mucus and other secretion/body fluids within the nasal cavity, resulting in the formation of a waste liquid. This waste liquid is drawn into an inlet at or near the terminus of the irrigation device, and transferred to a waste reservoir. The waste liquid can be drawn in using a pump that is in fluid communication with the waste reservoir. In some embodiments a vacuum or reduced pressure is applied to the waste reservoir (for example, using a pump, squeeze bulb or bottle, etc.), which in turn draws the waste fluid into the waste reservoir. The waste reservoir can be transparent to permit a user to readily determine the extent to which it is filled with waste fluid. The waste reservoir can be removable. Such a removable reservoir can be cleaned and returned to the nasal irrigation in some embodiments. In other embodiments the waste reservoir is disposed of after use.

In some embodiments waste fluid is withdrawn as the irrigation fluid is applied. These operation can be initiated at the same time, or one can be initiated before the other. For example, application of the irrigation fluid can be started, followed by initiation of waste fluid collection while irrigation fluid continues to be dispense. Alternatively, waste collection can be initiated prior to application of the irrigation fluid. In some embodiments application of the irrigation fluid and waste collection do not overlap. For example, irrigation fluid can be applied and waste fluid collection initiated after application of irrigation fluid has halted. In such an embodiment waste fluid collection can be started immediately on cessation of application of the irrigation fluid, or can be initiated after a suitable waiting period (e.g. to allow time for mucus to thin and/or applied pharmaceutical compounds in the irrigation fluid to act). The nasal irrigation system can include an indicator (e.g. a display or sound device) that indicates the status of nasal irrigation device during such a process in order to aid the person applying the irrigation in this process. For example, such an indicator can provide a timer, information related to whether or not the irrigation fluid is being dispensed, rate and/or volume of irrigation fluid dispensing, whether or not waste fluid collection is active, status of the irrigation fluid reservoir, and/or status of the waste fluid reservoir. In some embodiments such a display is provided on the attention focusing feature.

The attention focusing feature can be manipulated during the nasal irrigation process in order to maintain the attention of the individual being treated. Towards that end the focusing feature can display a pattern of lights, display a video or other animation via an electronic display, and or emit sounds. In some embodiments these displays and sounds are controlled by the person administering treatment, and can be used as positive reinforcement.

As noted above, the irrigation device of a nasal irrigation system can be removable and/or replaceable. This permits specific irrigation devices to be used with specific individuals, thereby avoiding cross contamination. In some embodiments the irrigation device is configured to be disposable.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A nasal irrigation device, comprising:
a pliant tube comprising a first lumen, a second lumen, a first aperture, a second aperture, and a terminus,
wherein the first aperture is positioned at the terminus and provides fluid communication between an external environment and the first lumen,
wherein the second aperture is oriented at or within 30 degrees of normal to the major axis of the first lumen and provides fluid communication between the second lumen and the external environment.

2. The nasal irrigation device of claim 1, wherein the pliant tube comprises an outer tube that at least partially encloses an inner tube, and wherein the second lumen is delimited by an inner wall of the outer tube and an outer wall of the inner tube.

3. The nasal irrigation device of claim 1, wherein the pliant tube comprises an outer tube that at least partially encloses an inner tube, and wherein the first lumen is delimited by an inner wall of the inner tube.

4. The nasal irrigation device of claim 1, wherein the first lumen and the second lumen occupy non-overlapping volumes within the nasal irrigation device.

5. The nasal irrigation device of claim 1, wherein the second lumen is in fluid communication with a source of irrigation solution.

6. The nasal irrigation device of claim 1, wherein the first lumen is in fluid communication with a source of negative pressure.

7. The nasal irrigation device of claim 6, further comprising a collection reservoir interposed between the first lumen and the source of negative pressure.

8. The nasal irrigation device of claim 1, wherein the pliant tube is dimensioned to lie within a nare or a nasal cavity of a human child.

9. A method of treating nasal congestion, comprising:
providing a nasal irrigation device comprising a double lumen pliant tube comprising a first lumen, a second lumen, a first aperture, a second aperture, and a terminus, wherein the first aperture is positioned at the terminus and provides fluid communication between nasal cavity and the first lumen, the second aperture is oriented at or within 30 degrees of normal to the major axis of the first lumen and provides fluid communication between the second lumen and the nasal cavity;
applying, via the second aperture, a volume of an irrigation solution laterally towards a wall of the nasal cavity; and
withdrawing, via the first aperture, a waste fluid from the nasal cavity.

10. The method of claim 9, wherein the double lumen pliant tube comprises an outer tube that at least partially encloses an inner tube, and wherein the second lumen is delimited by an inner wall of the outer tube and an outer wall of the inner tube.

11. The method of claim 9, wherein the double lumen pliant tube comprises an outer tube that at least partially encloses an inner tube, and wherein the first lumen is delimited by an inner wall of the inner tube.

12. The method of claim 9, wherein the first lumen and the second lumen occupy non-overlapping volumes within the nasal irrigation device.

13. The method of claim 9, wherein the waste fluid is directed to a collection reservoir.

14. The method of claim 9, wherein the volume of the irrigation solution is dispensed as a droplet spray.

15. The method of claim 9, wherein the step of applying the irrigation solution is performed over a first time period and the step of withdrawing the irrigation solution is performed over a second time period, and wherein the first time period and the second time period at least partially overlap.

16. A nasal irrigation system, comprising:
an irrigation nozzle comprising a double lumen pliant tube comprising a first lumen, a second lumen, a first aperture, a second aperture, and a terminus, wherein the first aperture is positioned at the terminus and provides fluid communication between an external environment and the first lumen, wherein the second aperture is oriented at or within 30 degrees of normal to the major axis of the first lumen and provides fluid communication between the second lumen and the external environment;
an irrigation fluid reservoir that is in fluid communication with the second lumen; and
a waste fluid reservoir that is in fluid communication with the first lumen.

17. The nasal irrigation system of claim 16, wherein the irrigation nozzle is replaceable.

18. The nasal irrigation system of claim 16, wherein the irrigation fluid reservoir comprises a pliant envelope configured to retain an irrigation fluid.

19. The nasal irrigation system of claim 16, further comprising a first pressurizing mechanism in communication with the fluid reservoir.

20. The nasal irrigation system of claim 19, wherein the first pressurizing mechanism comprises a mechanism that applies mechanical pressure to the irrigation fluid reservoir.

21. The nasal irrigation system of claim 16, further comprising a second pressurizing mechanism in fluid communication with the waste fluid reservoir.

22. The nasal irrigation system of claim 16, wherein the irrigation nozzle further comprises a third lumen, wherein the third lumen comprises a third aperture oriented at or within 30 degrees of normal to the major axis of the first lumen and providing fluid communication between the third lumen and the nasal cavity, and wherein the third lumen is in fluid communication with the irrigation fluid reservoir.

23. The nasal irrigation system of claim 22, wherein the second lumen and the third lumen are in fluid communication with a common channel, and wherein the common channel is in fluid communication with the irrigation fluid reservoir.

24. The nasal irrigation system of claim 16, further comprising an attention focusing feature positioned to distract a patient to which the nasal irrigation system is applied while the nasal irrigation system is in use.

25. The nasal irrigation system of claim 24, wherein the attention focusing feature comprises a visual target at least in part configured as a face.

26. The nasal irrigation system of claim 24, wherein the attention focusing feature comprises a visual target comprising one or more lights.

27. The nasal irrigation system of claim 24, wherein the attention focusing feature emits sound.

28. The nasal irrigation system of claim 24, wherein the attention focusing feature comprises an electronic display.

29. The nasal irrigation system of claim 16, wherein the irrigation fluid reservoir further comprises an irrigation fluid.

30. The nasal irrigation system of claim 29, wherein the irrigation fluid further comprises a pharmaceutical compound.
